(19) United States
(12) Patent Application Publication
(10) Pub. No.: US 2007/0255177 A1
(43) Pub. Date: Nov. 1, 2007

(54) Devices and methods for collecting oral samples of enriched serous fluid

(76) Inventor: Allan D. Pronovost, San Diego, CA (US)

Correspondence Address: FOLEY & LARDNER LLP, 111 HUNTINGTON AVENUE, 26TH FLOOR, BOSTON, MA, 02199-7610, US

(21) Appl. No.: 11/796,488

(22) Filed: Apr. 27, 2007

Related U.S. Application Data
(60) Provisional application No. 60/795,536, filed on Apr. 27, 2006.

(51) Int. Cl. A61B 5/00

(52) U.S. Cl. 600/573

(57) ABSTRACT

The present invention relates to serous fluid collection devices, and methods of using the same to collect orally samples of enriched serous fluid suitable for a target diagnostic medical procedure. Sample collection devices include at least one sample inducing region adapted to induce a flow of enriched serous fluid, and at least one sample collection member, such as a sponge, adapted to collect a suitable sample volume as determined by the target assay. The sample inducing region is adapted to induce blood equivalent levels of serous fluid flow by one or more chemical, physical, or biological induction means. In some embodiments, means are optionally provided to visually mask erythrocytes contained within the collected sample.

### CROSS-REFERENCE TO RELATED PATENT APPLICATIONS

This application claims priority to U.S. Provisional Patent Application No. 60/795,536, filed on Apr. 27, 2006. The entire teachings of the above application are incorporated herein by reference.

### FIELD OF THE INVENTION

The present invention relates generally to the field of immunological testing and more particularly to the collection of samples suitable for such testing obtained from the oral cavity.

### BACKGROUND OF THE INVENTION

There are numerous methods and devices known in the art for the collection of an oral fluid. For example, U.S. Pat. No. 5,335,673 describes the use of an untreated cellulosic absorbent pad for the collection of oral fluid. After collection of the oral fluid, the absorbent pad is placed in a container with cap containing a hypertonic solution of alkaloid metal salts, or alkaline earth metal salts and a preservative for temporary storage. The collection device is then mailed to a central lab for immunoglobulin testing by Enzyme-Linked ImmunoSorbent Assay (ELISA). U.S. Pat. No. 5,103,836 describes alkaloid metal salts or alkaline earth metal salts pre-impregnated onto a pad. U.S. Pat. No. 5,830,154 describes a collection device for oral fluid involving a two-stage device: one portion is used to collect oral fluid by expectoration, followed by interface with a second portion containing preservative and hypertonic solution of alkaloid metal salts or alkaline earth metal salts. U.S. Pat. No. 5,022,409 describes the use of an oral rinse solution compromising a hypertonic solution of alkaloid metal salts or alkaline earth metal salts. U.S. Pat. Nos. 5,477,937; 5,573,009; and 5,339,829 all describe a simple syringe like device for collection of oral fluid.

Some patents describe integration of a collection device with assay means: For example, U.S. Pat. No. 6,303,081; and U.S. Published Application No. 20020155029 describe an apparatus for collection of oral fluid for lateral flow chromatography or variants, or components thereof. The device contains a lateral flow chromatography strip and a sample collection device integrated into a single unit and is described for use for oral antibody detection. International Application Publication No. WO 0702889 describes a similar device, but also provides the means to retain the original sample for drugs of abuse confirmation testing. U.S. Pat. No. 5,830,410 describes a collection device comprising a syringe used to draw up the sample. Each of the aforementioned references is hereby incorporated by reference in its entirety.

In each of the above cases, the collection of oral fluid is essentially a passive event. A device, such as a wick, is placed in the mouth and collects oral fluids that happen to be in the vicinity of the wick in the oral cavity—typically saliva.

In the above references, hypertonic salts, preservatives, and stabilizers are additives designed to preserve the sample for transport to a laboratory. Thus, the methods and devices referred to in some of the above references involve sample collection and transport only. Detection is accomplished subsequently, following submission of the collected sample to a laboratory using Western Blot or ELISA. Later designs involved integration of components together for point-of-care testing. Unfortunately, due to the random occurrence of antibodies in the oral sample, use of the above collection devices whether in a laboratory or at point-of-care, is subject to a high false negative rate. Accordingly, such techniques are not ideally suited for screening applications, such as for AIDS, a particular concern in third-world countries where epidemiological control is paramount.

U.S. Pat. No. 6,926,681 describes the method and system for performing micro abrasion and suction using a vacuum generator. U.S. Pat. No. 6,673,081 describes the use of sterile fluid, i.e., water, to achieve dermal abrasion in combination with air pressure as a means of sample collection. The wash fluid is analyzed. U.S. Pat. No. 6,540,757 describes the use of a pressure cartridge system for dermal abrasion also employing a vacuum means. U.S. Pat. No. 6,869,611 describes the means for effecting superficial chemical skin peels through the use of salicylic acid. All of these references relate to non-oral applications. Each of the aforementioned references is hereby incorporated by reference in its entirety.

A variety of other means are described for the use of blood and/or interstitial fluid, which involves trace amounts of fluid between cells. Examples of these are described in U.S. Pat. Nos. 7,016,713; 7,001,343; 6,904,301; 6,712,776; 6,602,205; and 6,537,243; among others. Collection of interstitial fluid can be accomplished through a variety of micro-sampling means mostly involving micro-lancets, or through reverse iontophoresis (electrical stimulation) means, or other micro-invasive blood sampling methods applied non-orally. U.S. Pat. No. 6,245,060 describes the use of laser light as a means for removal of the stratum corneum to obtain interstitial fluid and blood non-orally. Each of the aforementioned references is hereby incorporated by reference in its entirety.

### SUMMARY OF THE INVENTION

None of the above references suggest that oral fluids have the same properties (or diagnostic potential) equivalent to, or correlatable to a blood or serous fluid sample, nor do any of the above references describe any means to obtain and enrich the serum and hence the immunoglobulin levels present in oral fluid. The presence of any antibodies in oral fluid samples collected by any of the above devices would be by chance, owing to the random contamination of the oral fluid. Such contamination may result in a saliva sample containing traces of crevicular fluid resulting from poor gum care and/or periodontal disease.

Saliva is a dilute fluid relative to blood and is produced by cellular plasma membrane ultrafiltration between the bloodvascular and saliva glands. The physiology of saliva production is well understood and studies have shown that saliva within the glands contains substantially lower concentrations of materials than found in blood and is relatively devoid of high molecular weight molecules present in blood. Saliva typically contains low molecular weight materials less than 20,000 daltons. Macromolecules like human serum albumin or immunoglobulins like IgG (140,000 daltons) are not present in glandular saliva. IgG and other high molecular weight substances have been reported however in oral fluid but their presence is not attributable to saliva but to crevicular fluid as the result of trace bleeding. Immunoglobulin levels in whole oral fluid are extremely low making the detection of IgG and other immunoglobulins in saliva difficult at best and certainly not predictive of or correlating with blood levels. Trace contamination is not a reliable factor for measurement of immunoglobulin status of individuals.

Beneficially, the present invention provides device and method for inducing flow of enriched serous fluid into the oral cavity and capturing a suitable sample volume thereof for performing a target assay. Collection of blood equivalent levels of serous-fluid orally is accomplished through chemical, physical, or biological induction means. The enriched samples preferably include blood-equivalent (or correlatable) levels of any analyte (or substance found in blood), allowing for screening without the complexity, complications, and risks associated with obtaining blood samples. Thus, the present device improves upon various patents, patent documents, and publications cited herein, which are hereby incorporated by reference in their entirety.

In one aspect, the invention relates to an oral serous fluid collection device adapted to collect an enriched sample of serous fluid from an oral cavity. The device includes a substrate configured for placement within an oral cavity. A sample collector is attached to the substrate and configured to collect a sufficient sample volume for performing a target assay. The device also includes at least one sample inducer disposed on at least one of the substrate and the sample collector. The sample inducer is configured to induce a flow of serous fluid from an oral surface.

In one aspect, the invention relates to a process for collecting an enriched serous fluid sample from a mammalian oral cavity. A sample inducer is placed against an adjacent surface of the oral cavity. The sample inducer induces a flow of enriched serous fluid from the oral surface. A sufficient sample volume of the induced, enriched serous fluid is then collected, the sample volume being sufficient for performing a target assay.

In another aspect, the invention relates to a serous fluid collection kit including a serous fluid collection device, sealable packaging for storing the serous fluid collection device, and instructions for using the device. The serous fluid collection device includes an oral serous fluid collection device adapted to collect an enriched sample of serous fluid from an oral cavity. The device includes a substrate configured for placement within an oral cavity. A sample collector is attached to the substrate and configured to collect a sufficient sample volume for performing a target assay. The device also includes at least one sample inducer disposed on at least one of the substrate and the sample collector. The sample inducer is configured to induce a flow of serous fluid from an oral surface.

In yet another aspect, the invention relates to a method of detecting an analyte or substrate in a mammal. A sample of serous fluid is obtained from the mammal using a serous fluid collection device. An analyte or substrate is detected in the obtained sample of serous fluid. The serous fluid collection device includes an oral serous fluid collection device adapted to collect an enriched sample of serous fluid from an oral cavity. The device includes a substrate configured for placement within an oral cavity. A sample collector is attached to the substrate and configured to collect a sufficient sample volume for performing a target assay. The device also includes at least one sample inducer disposed on at least one of the substrate and the sample collector. The sample inducer is configured to induce a flow of serous fluid from an oral surface.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1A and FIG. 1B are schematic diagrams respectively illustrating a top view and side elevation view of an exemplary embodiment of an oral sample collection device constructed in accordance with the present invention.

FIG. 2 is a schematic diagram illustrating a side elevation view of an alternative embodiment of an oral sample collection device including a mechanical abrasion surface constructed in accordance with the present invention.

FIG. 3 is a schematic diagram illustrating a side elevation view of yet another embodiment of an oral sample collection device also constructed in accordance with the present invention.

FIG. 4 is a flow diagram of a method of collecting an oral serous fluid sample in accordance with the present invention.

### DETAILED DESCRIPTION OF THE PREFERRED EMBODIMENTS

The present invention provides devices and methods for inducing and orally collecting samples of serous fluid suitable for use in any assay. Production of serous fluid is induced within an oral cavity through one or more of chemical, physical, and biological induction means. Preferably the oral samples include blood equivalent levels of serous-fluid, which can be used in a biomedical procedure to assay for an infection or to assist in the diagnosis of a subject with a disease state. Oral sample collection is preferred over blood collection in many instances.

It is a primary advantage in developing countries to collect a sample for analysis for screening, diagnosis, or monitoring purposes that is devoid of a discernable level of erythrocytes. Collection of a blood sample in said countries is often prohibitive for religious, cultural and/or ethical reasons; not to mention for health and containment reasons as well, especially with HIV infections. Under such conditions it would be ideal to obtain a blood-equivalent sample. The ideal collection means would afford the following properties to the collection method. The collection site would be non-visible, so as not to be seen by others. Collection would need to be self-administered without the aid of other individuals, and collection would need to be simple for third world individuals. The method should assume high compliance. The method should apply to any analyte of medical importance and should provide a sample containing blood-equivalent (or correlatable) levels of analyte whether the analyte is low or high molecular weight in nature. The method would also apply to any natural substance found in blood, serum, or plasma and the ability to measure it reliably and quantitatively in oral fluid enriched with serous fluid. The method should be minimally invasive, contain minimal levels of blood cells or be processed and/or treated in such a way as to disguise, disrupt, or remove blood cells above a visual threshold. The method may allow for micro-capillary disruption without overt bleeding orally.

In this invention, the collection of blood-equivalent levels of serous fluid orally is accomplished without significant co-contamination of the oral sample by erythrocytes, i.e., induction of observable bleeding. The method is designed to rapidly collect a substantial amount of serous fluid (i.e., more than about 1 micro liter, but generally less than about 200 micro liters per collection event) dispersed in the oral fluid.

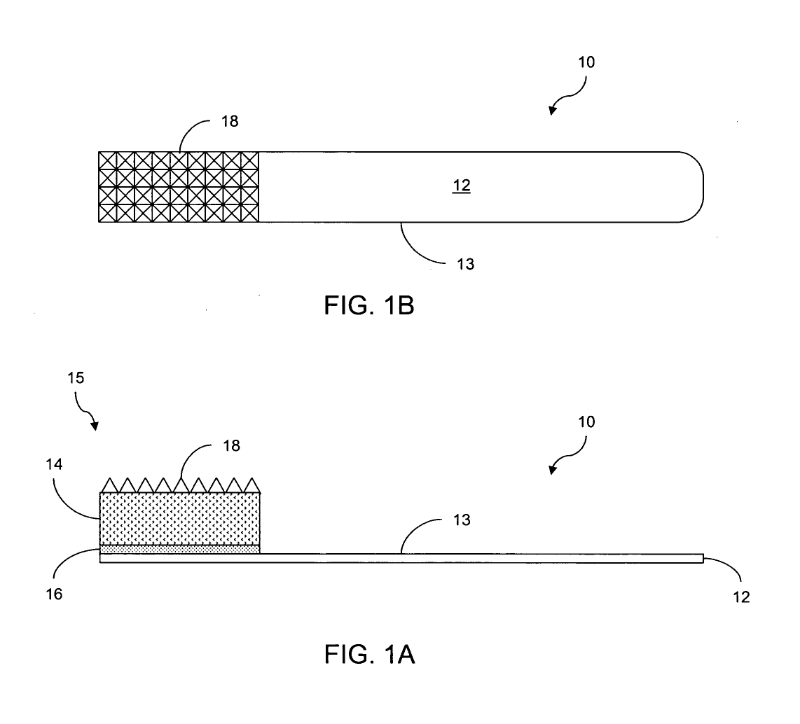

In its most simplistic embodiments, the invention provides a serous fluid collection device that includes a substrate having a serous fluid inducing region, and a collection region. Referring to FIG. 1A and FIG. 1B, an oral sample collection device 10 includes a substrate including a sample collection end 15 disposed at a proximal end of the device 10. As shown, the substrate 12 can be planar and include an elongated distal end serving as a handle 13 for the device 10. The sample collection end 15 includes a sample-retaining member 14 attached to the substrate 12. Attachment can be accomplished using one or more of chemical bonding, thermal bonding, and mechanical fasteners. As shown, the sample-retaining member 14 is attached to the substrate 12 using a layer of adhesive 16. The adhesive can be a liquid or paste adhesive manually applied to on or more mating surfaces of the sample retaining member 14 and the substrate 12 during fabrication of the device. Alternatively or in addition, the adhesive can be a double-sided adhesive tape.

The device also includes a sample inducing region adapted to induce flow of serous fluid prior to collection by the sample retaining member 14. In some embodiments, the sample-inducing region is applied to at least a portion of at least one outer surface of the sample-retaining member 14. In the exemplary illustrated embodiment, the sample-retaining member 14 is a porous material, such as a sponge, formed into a rectangular shape. The sample-inducing region 18 is located along a top surface of the sponge 14. In some embodiments, the overall shape of the device can resemble a standard toothbrush according to its size and approximate shape, making it well adapted for oral use.

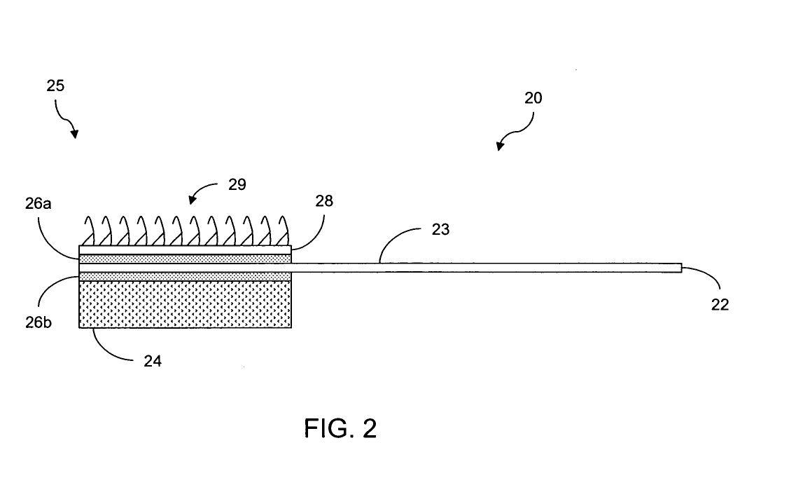

An alternative embodiment of a sample collection device 20 is illustrated in FIG. 2. The device 20 includes a substrate 22 including a sample collection end 25 disposed at a proximal end of the device 20. Once again, the substrate 22 can be planar and include an elongated distal end serving as a handle 23 for the device 20. The sample collection end 25 includes a sample-retaining member 24 attached to one surface of the planar substrate 12. Attachment can again be accomplished using any of the means described above in reference to FIG. 1, such as a layer of adhesive 26b.

The device 20 also includes a sample inducing region 28, 29 adapted to induce flow of serous fluid prior to collection by the sample retaining member 24. In an alternative arrangement, the sample-inducing region 28, 29 is attached to a different surface of the planar substrate 22, opposing the sample-retaining member 14. In general, the sample inducing region 28, 29 can include any suitable sample inducing means. As illustrated, the sample-inducing region includes a hook portion of a hook and loop fastening system. A multitude of hooks 29 are disposed along one side of a planar surface, such as a tape or ribbon 28. The opposite side of the ribbon 28 is attached to the substrate 22 using any suitable means of attachment, such as an adhesive layer 26a, as shown.

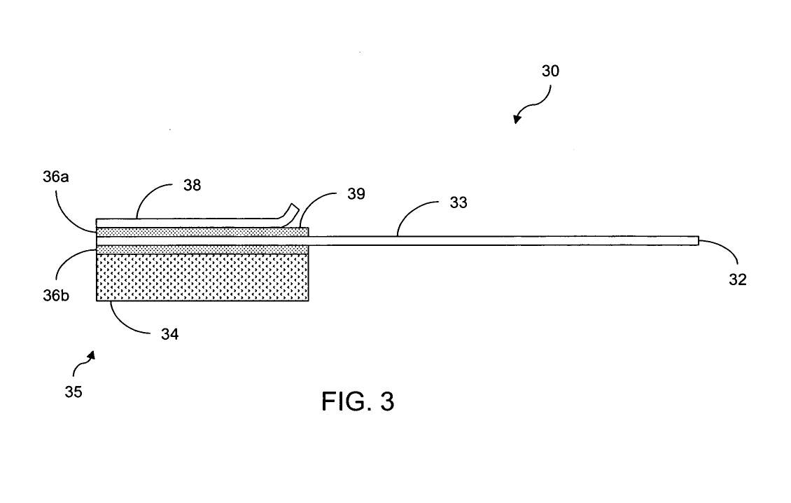

Yet another alternative embodiment of a sample collection device 30 is illustrated in FIG. 3 is a variant of the device described in relation to FIG. 2. The device 30 includes a substrate 32 including a sample collection end 35 disposed at a proximal end of the device 30. Once again, the substrate 32 can be planar and include an elongated distal end serving as a handle 33 for the device 30. The sample collection end 35 includes a sample-retaining member 34 attached to one surface of the planar substrate 32. Attachment can again be accomplished using any suitable means of attachment, such as a layer of adhesive 36b.

In the exemplary alternative arrangement, the sample-inducing region consists of an adhesive layer 36a temporarily covered by a removable backing 39, such as a foil, paper, plastic, or biaxially-oriented polyethylene terephthalate, commercially available from DuPont Company under the trade name MYLAR. As illustrated, the sample-inducing region can be mounted to an opposite surface of the substrate 32 opposite to the sample-collecting region 34. The removable backing 38 is removed prior to use, and the exposed surface 39 of the adhesive layer 36a is placed against a surface of the sample region within the oral cavity. The adhesive layer 36a temporarily adheres to the skin surface, such that when the device 30 is pulled away from the surface, one or more layers of cells are removed with the device 30.

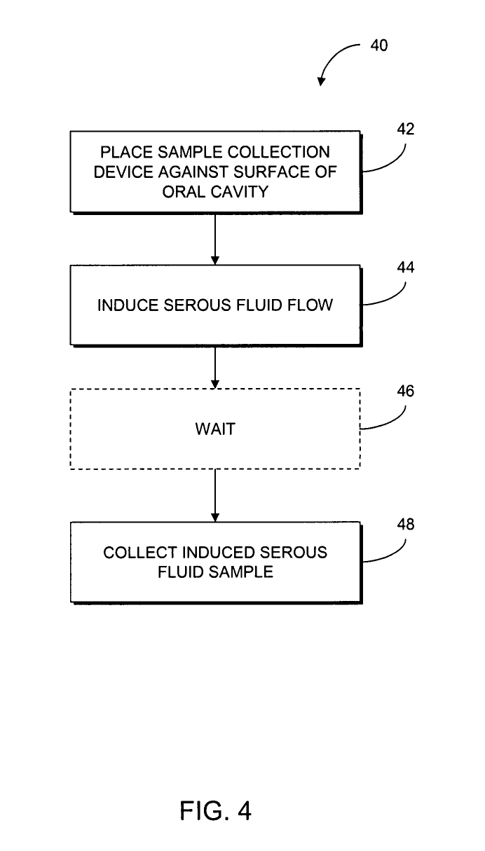

FIG. 4 an exemplary process 40 for using the sample collection device 10 to obtain a suitable sample volume of serous fluid for an intended assay. The process can be initiated by an operator through insertion of at least a proximal end of the sample collection device 10 (FIG. 1) into an oral cavity, such that the sample-inducing region 18 (FIG. 1) is urged against a sample production surface within the oral cavity (Step 42). The process 40 can be self-performed, in which the operator is an individual collecting a sample without assistance, or an assisted process in which another, such as a clinician obtains the sample.

The operator manipulates the sample-inducing region 18 to stimulate or induce production of serous fluid at the sample production surface (Step 44). Manipulation can be simply placing the sample-inducing region 18 into contact with the sample collection surface and maintaining such contact for a predetermined period of time. This technique is generally used for a chemically or biologically treated sample-inducing region 18. Alternatively or in addition, manipulation can be accomplished by rubbing the sample-inducing region 18 of the device 10 when placed in contact with an oral surface to abrade the surface.

The operator next brings the sample-retaining member 14 into contact with the sample production surface to capture a sample of the induced serous fluid flow (Step 48). In some embodiments, the operator waits for at least a minimum delay period (Step 46) between induction of the serous flow (Step 44) and collection of the induced sample (Step 48).

In some embodiments, the sample-inducing region 18, 28, 38 includes effective amounts of one or more compounds that induce serous fluid flow at the site of application, where the induced serous fluid flow is directed into the collection region. Alternatively or in addition, the sample-inducing region 18, 28, 38 includes a cell-removing region, for debridement of tissues. In various embodiments, the sample-inducing region 18, 28, 38 and sample retaining or collection region 14, 24, 34 are all discrete regions on the substrate 12, 22, 32, although in currently preferred embodiments, they are contiguous, overlapping, interspersed or otherwise co-localized. The properties of the various layers are described in turn.

Induction of serous fluid flow is accomplished by a variety of chemical, biological or physical compounds and methods. Certain compounds or methods effect local debridement of one or more cellular layers, but some will induce serous fluid flow without cellular disruption. Cellular removal is indicated when larger fluid samples are needed.

A variety of chemical means may be employed for induction of serous fluid to yield an oral sample enriched in serous fluid to aid detection of analytes at a level equivalent to or correlatable to a blood sample. Chemical induction methods of serous fluid from the disrupted or non-disrupted buccal cell layer of the inner cheek (or from the gums) include the use of dilute organic acid solutions, such as acetic acid, malic acid, citric acid or the like; and/or the use of urea. These chemicals may be applied directly to the buccal cell layer as powder, or as liquid, or as a solid dosage form, i.e., pill, or may be impregnated onto a collection pad.

These chemical agents may aid serous flow by establishing a chemical or osmotic gradient between the dermal layer and the collection pad matrix interface, wherein fluid flow will be driven osmotically from low (within the disrupted skin layer) to high (within the pad) concentrations. This will proceed until equilibrium was achieved.

Alternatively, a moisture gradient may be established through use of appropriate chemical desiccating agents. Incorporation of appropriate micro-desiccants such as inert ceramic micro-particle desiccants, like ATS (Engelhard), ceramic zeospheres (3M), silica gel or silica gel containing desiccating papers such as Drikette Desiccant Paper (Multisorb Technologies, West Seneca, N.Y.), or hydrophilic silica nano-particles such as M5 or EH-5 (Cabot), or AEROSIL 200 Pharma (deGussa), or the like, into collection pads will help unidirectional fluid flow along the gradient from the disrupted skin layer to the collection pad.

Alternatively an absorbent polymer layer can be established through the use of water absorbing powders that form a hydrogel or fluid imbibed layer upon hydration. Such swellable materials, available in powdered form, include the Sephadex Series (Pharmacia), comprised of cross-linked agorsose granules; or polyacrylamide or bis ethelene acylamide or sodium polyacrylic acid; xanthum gums; carboxymethylcellulose; polyvinyl alcohol; or the like or super-absorbent starch co-polymers such as poly(2-propenamide-co-2-propenic acid), available as the sodium or potassium salt or starch co-polymers such as (2-propenamide-co-2-propenoic acid, sodium salt), which are available from GPC. The latter materials have swell ratios of >130 to 600+ times their weight. These can be coated as a gel monolayer to adhesive attached to a device.

Chemical induction agents may also afford specific physical properties to further aid in serous fluid induction from the disrupted buccal cell layer (or the gums). Chemical induction agents which exhibit an exothermic reaction upon primary hydration in the mouth may be utilized to induce inflammation, swelling, and chemical burn if needed, at the site of application. Application may be done through incorporation of the exothermic chemical agent onto a sample collection pad or through direct application of the chemical as powder or liquid, between the cheek and the gums, or through the placement of a solid dosage form, i.e., pill, containing the pelletized chemical, between the cheek and the gums. Exothermic chemical agents requiring hydration with water (as found orally) include calcium oxide (quicklime), calcium sulfate, calcium carbonate, calcium chloride (as used in heat packs), or sodium acetate plus water. The latter would be sealed within an insertable pouch to achieve a temperature of 130 degrees centigrade upon activation.

Serous fluid induction from the buccal cell layer may also be achieved both chemically and physically through the use of capsaicin containing compounds with a Scoville heat unit greater than 5,000. The use of pharmaceutically processed capsaicin will induce serous flow, especially if coupled with dermal abrasion, through vasodilation and/or inflammatory means. Capsaicinoids are natural substances that produce a burning sensation in the mouth, causing the eyes to water and the nose to run, and even inducing perspiration. The primary capsaicinoid, capsaicin, diluted 1:100,000 in water, will produce dermal blisters.

Capsaicinoid content is measured in parts per million and one part per million is equivalent to 15 Scoville units. Pure capsaicin has a Scoville heat unit score of 16 million. Capsaicin is the common name for (8-methyl N-vanillyl 6nonamide). Other useful capsaicinoids include dihydrocapsaicin, nordihydrocapsaicin, homodihydrocapsaicin, and homocapsaicin. Synthetic topical pharmaceutical preparations of capsaicin of particular use are Axsain, & Zostrix.

In the use of exothermic chemical inducers and the use of capsaicinoids it may be advisable and necessary to add chemicals that would counteract pain locally. This may also lessen the effect of abrasion. Local anesthetics may be applied before, after, or during the collection process and chemical based anesthetics ma be applied to a powder, solid dosage form, liquid, or integrated into a collection device matrix. Examples of anesthetics are menthol, camphor, lidocaine, prilocaine, benzocaine, butacaine, cyclomethycaine, dibucaine, or tetracaine, or the like.

Serous fluid may also be induced orally through the use of the chemical salicylic acid, sodium salicylate or other acetylated on non-acetylated salicylates dispensed as above or incorporated into a collection pad.

A variety of vasodilators may also be applied directly to the buccal cell layer of the cheek or the gums. Application of vasodilators will induce skin permeability, and enhance exudate flow. Vasodilators may be used in liquid, powder, or tablet form and may be used alone or coated onto a collection pad. Examples of vasodilators include histamine, bradykinin, 5-HT, PAF, prostroglandins, and the like.

Parasympathetic nervous system stimulators mimicking the effect of acetylcholine may be used for induction of serous fluid and/or saliva. Pilocarpine stimulates large amounts of saliva and is available orally in tablet form. SALAGEN® tablets (5 mg), containing pilocarpine hydrochloride are used to treat xerostima. Pilocarpine may be used in pill form to induce serous flow or may be added to other formulations.

Calcium chelators such as EDTA, or citric acid, either applied alone or as part of a collection pad may be particularly effective at inhibiting fibrin polymerization orally in order to continue and maintain serous flow orally without blood clot formation. This will allow for unimpeded serous fluid flow.

A variety of biological means may be used for the induction of serous flow orally from the buccal cell layer (or from the gums). This may be accomplished through the use of enzymes as the means for enzymatic tissue digestion. Single enzymes, or cocktails of enzymes, may be employed alone or in conjunction with other chemical or physical induction means. Suitable enzymes include serine proteases, such as chymotrypsin, or elastase, trypsin, proteinase K, papain, or other enzymes such as serine proteases hydrolyze either esters or peptide bonds, which allow dissolution of connective tissue. The combination of collagenase and proteinase K, or the use of papain with urea is particularly effective.

A variety of physical means may be used to achieve dermal abrasion of the buccal cell layer or the gums orally, as the means for serous fluid induction. These include the use of aluminum oxide powders, impregnated onto suitable supports. Aluminum oxide powders with a high grit size (18-100) are particularly effective at dermal abrasion. Calcined alumina oxide powders may also be employed to redefine the shredding technique. Aluminum oxide powders can be coated directly onto a collection means, i.e., stick or paddle, containing double stick adhesive, or directly onto sponges, cloths, tapes, paper, or other appropriate support media. Alternatives to aluminum oxide include silicon carbide, zirconia alumina, crushed glass, crocus; or the like. Plastic or metal woven sheets of low grit size such as Abranet (MIRKA) 40-400 grit mesh cloth, or 16-600 grit abrasive cloth sheets from Suzhou Shi De Enterprise Development Co, or equivalent can be employed.

Aluminum oxide coated abrasive sponges of 16 to 600 grit size are available through Suzhou Shi De Enterprise Development Co., in Jiangsu Province, or Shengshi Abrasive Co. in Hubei Province, China. Abrasive cloth sheets made of aluminum oxide, zirconia alumina, or silicon carbide C with a grit size of 16 to 600 are available in the hard or soft form (Suzhou She De Enterprise Development Co).

Alternatively, calcined alumina powders of defined grain shape, purity, and uniform size, may be obtained from Saint-Gobain Ceramics and Plastics, Inc., Worcester, Mass. Suitable grades of calcined alumina include Norton Industrial grade Precision Alumina, code 7920 in sizes from 3 to 25 microns. Non-calcined fused aluminum oxide general purpose grades may also be used; examples include Saint-Gobain 9410 E 111 H (average size is 15 microns), 9407 E70 (4.5 microns), or 7987 E266 (<10 microns). Calcined or non-calcined alumina powders may be attached directly to a plastic or wooden collection device through the use of double stick adhesive.

Another suitable physical means for induction of serous fluid orally through dermal abrasion would involve the use of the hook component used in “hook and loop” fasteners such as VELCRO brand MVA 8 Hook, or DuraGrip Fasteners (Ferro Corporation). Additionally, micro-machined or laser sculpted micro-sculptures on metal surfaces or films can be employed. The latter involves the fashioning of delicate, yet durable metal projections to yield ultra strong hooks for dermal abrasion. Examples by way of reference include the Surfi-Sculpt process, developed at The Welding Institute (TWI in Great Abbington, Cambridge, UK), or the use of laser-ablated thick metallic films.

Concerning hook and fasteners suitable for this purpose, U.S. Pat. Nos. 2,717,437; 4,775,310; 4,794,028; 4,872,243; 5,315,740; 5,339,499; 5,260,015; 5,518,795; 5,744,080; and 5,802,676 are hereby incorporated by reference. Hook fasteners may be composed of woven nylon or other resins, blended nylon, or molded nylon and will have a min-max tensile psi strength range of 4.5 up to 20.2, and a longitudinal min-max shear psi range of 5.6 to 40.5.

Other physical methods for dermal abrasion may include the use of files, graters, brushes, or pads to aid in buccal cell layer removal. Such means can involve the stripping of the dermal layer, scraping of the dermal layer, tearing of the dermal layer, ripping of the dermal layer, peeling of the dermal layer, in addition to other chemical, or biological induction means.

Another physical means for the induction of serous flow orally will involve removal of the buccal cell dermal layer through the application of water resistant adhesives. Adhesives that are capable of adhering to the wet skin, thereby forming a strong bond and subsequently being removed with removal of the buccal cell layer, will be particularly useful. Adhesives may be applied in one of two manners for cell removal. Method one will involve the addition of liquid adhesive, (such as cyanoacrylate) to an absorbent pad, placing said pad in the mouth between the cheek and the gums before the adhesive sets, allowing the bond to occur (up to 30 seconds), and removal of the pad with the dermal layer attached resulting in exposure of underlying layers and subsequent serous exudation. Palm Labs Ire. (Hilton Head Sc) liquid Turbo Fuse 15-190 or Turbo Fuse gel 07-200 is a rapid hardening ethyl cyanoacrylate adhesive designed to polymerize instantly by absorbing surface moisture.

Another method will involve the removal of a release liner from a solid adhesive attached as a double stick adhesive to a collection device, i.e., stick, paddle, hence allowing adherence to the buccal cell dermal layer, followed by pressing the cheek from the outside to assure adherence, and subsequent removal of the adhesive backed paddle and attached buccal cell dermal layer resulting in exposure of underlying layers and subsequent serous exudation. Examples of suitable double sided adhesives useful for dermal abrasion include the ARCARE® Healthcare adhesives 6689, 7137,7261, 7936, 7717, 7806, 7922, 8311, 8383, 8570, 8651, 8881, 8890, and 90374 of Adhesives Research (Glen Rock, Pa.) in addition to the other sources.

Any of the physical methods described herein may be coupled with the biological and/or chemical means noted to facilitate heavy serous fluid flow orally without significant bleeding.

A variety of materials can be used for collection device pads. Pads may be of four forms: absorptive and inert; absorptive and organic (non-inert composition); or adsorptive, being either inert or non-inert.

Examples of absorptive inert materials include high density hydrophilic polypropylene (Porex), polyurethane foam ((Shengshi), GF-A or GF-B glass fiber membranes (Whatman); aluminum oxide filters (Anodisc, Whatman); polycarbonate (Osmonics), polyester, PVDF, polyolefin (Filtrona), PTFE, or other membrane filters and materials (Steriltech, Whatman Nuclepore) or the like, composed of non-organic inert plastic, glass, aluminum, or other inert non-bibulous materials. Absorptivity is attributable to porosity of the three dimensional matrix.

Examples of absorptive non-inert materials include cellulosic membranes and cellulosic blends (Whatman, Schleicher and Schuell), which are absorptive due to both composition and porosity, i.e., bibulous, and may require pretreatment with surfactants or proteins to minimize analyte binding.

Thirdly, examples of adsorptive inert materials include Zeolite films or Zeolite-containing media, silica gel papers like Drikette, nylon membranes such as Biodyne B, Biodyne Plus (Pall).

Examples of adsorptive non-inert media include nitrocellulose membranes such as Biotrace NT (Pall), cellulose nitrate (Sartorius), cellulose nitrate (whatman), and the like. In the latter case, analytes adsorb directly to the media unless the media pretreated to render it inert to remove innate binding properties.

Absorptive or adsorptive media selection, whether inert or non-inert, is dependant upon the application. Media may be of any thickness, and may be treated to be bibulous or non-bibulous, chromatographic or non-chromatographic, analyte binding or non-analyte binding, or the like, depending upon the collection device function and the analyte detection means. The device, containing the collection device media, may be selected and designed to be independent of analyte detection or participative in the analyte detection process. Analyte detection may be accomplished by any method known in the art and not limited to chemical, biochemical, immunochemical, physical, or bioassay detection means by visual, non-visual, instrumented, or non-instrumented means.

The presence of trace levels of erythrocytes in the induced oral sample, due to micro-capillary disruption, may be dealt with by a variety of means. In developing countries it is imperative that the sample collected look “non-bloody” by visual recognition means.

The simplest method to mask the presence of trace amounts of blood cells is to place the collection pad into a tube containing a solution (as part pf the next step in an assay, wherein the collection device is placed in said solution to remove the collected fluid from the collection device and provide sufficient volume to run an assay). The solution may contain a red dye (red dye #40) at a level sufficient to mask heme levels. Alternatively or in addition, the collection pad matrix, which is used to collect the sample, can be colored, or dyed to provide a red hue, or made of red colored material. It is also possible, in either case, to use another color, such as a primary color contrasting that of red. For example use of the color blue, would yield a purple hue, and yellow would yield an orange hue when wither is stained with red blood cells.

Another means to disguise the presence of erythrocytes in the oral sample is to lyse the red blood cells. This can be accomplished by a variety of means known in the art, including the use of hypotonic solutions to disrupt cells, use of surfactants to dissolve cell membranes, or by chemical means. Hemoglobin released as the result of such a process, can be disguised through chemical oxidation using sodium hypochlorite, sodium percarbonate, sodium carbonate, or the like. Oxidation can be accomplished by a variety of known chemical means including, but not limited to, hydrogen removal, pH control, single electron transfer electron removal, per-oxygen oxidation, use of heavy metals, or other means. Alternatively the hemoglobin can be reduced resulting in oxygen removal with change from a red to blue-green tint. Chemical reducing agents may be single-electron transfer, electron donor reducing agents, e.g., lithium, hydride complex reducing agents, e.g., lithium aluminum hydride; Lewis acid hydride donor reducing agents, e.g., sodium borohydride; hydrogen, as a reducing agent through pH control; use of heavy metal reducing agents; or the like.

### EXAMPLES

A variety of collection devices can be designed to induce the collection of serous fluid orally. The amount of serous fluid to be induced in the oral sample is dependent upon the sample volume needs of the assay, the volume of serous fluid needed to be released, and the relative concentration of analyte in serum (blood). Analytes present in blood at a high concentration, such as glucose (mg/dl), will require less serous fluid induction into oral fluid versus analytes at a low concentration in blood, such as PTH (pg/ml). The volume of serous fluid to be induced is directly proportional to the amount of serous fluid needed in the unprocessed oral sample. The volume of serous fluid needed in the oral sample is a factor, one of several, determining the methods or combination of methods of induction to be employed.

Physical, or chemical, or biological induction methods may be used alone if the volume of serous fluid induced is adequate. Alternatively, combinations of physical and chemical, physical and biological, chemical and biological, or the combination of all three may be used. When a combination of two induction means is utilized, say physical and chemical, the two methods may be used simultaneously, in tandem, or in reverse order. As example, physical abrasion of the skin with hook fasteners or aluminum oxide cloth may be followed by the use of an organic acid to induce flow or alternatively an exothermic chemical reaction may be used, first to induce swelling and edema, followed by physical abrasion to release the built up fluid. This is especially applicable to the dermal layers of the cheek, which exhibit high degrees of vascularization (i.e., points of fluid collection, i.e., blushing). Other mucosal sites may be used for induction of serous fluid. These include the skin (behind clothing), the underarms, inner-thigh, vaginal area, intranasally, etc.

It is understood by those in the art that the following examples describe specific embodiments for exemplary purposes but are not all-inclusive or limited by description. Any single method or combination of methods, as described herein, whether they be physical, chemical, or biological can be employed, depending upon the serous sample requirement for oral fluid, based on the analyte need.

### Example 1

Referring again to FIG. 1, an exemplary multilayered sample collection device 10 includes a sample retaining member 14 coated on one side with an abrasive forming a roughened surface 18. The sample-retaining member 14 is formed from a yellow urethane foam sponge 14, coated on one side with a 40-grit aluminum oxide compound, forming the roughened surface 18. The foam sponge 14 is formed from a 6.4 mm (about 0.25 inch) thick foam sheet stock cut into a 7×20 mm block (about 9/32× 25/32 inches). The foam sponge 14 is adhered to a top surface of a proximal end of the substrate 12. The substrate 12 is formed from a 0.5 mm (about 20 mills) thick plastic strip of polyethylene terephthalate (PET), commercially available from Polyester Converters of Fullerton, Calif. The substrate 12 is formed in a substantially rectangular shape of about 7×120 mm (about 9/32× 4/75 inches). The foam sponge 14 is adhered to the top surface of the proximal end of the substrate 12 using an adhesive layer. The adhesive layer 16 is dimensioned to substantially cover the adjacent surface of the sponge 14 (i.e., 7×20 mm (about 9/32× 25/32 inches)). In the exemplary embodiment, the adhesive 16 is a piece of acrylic, non-sensitizing medical grade double stick adhesive 16, such as HY-3 High MVTR commercially available from Adhesive Research, Inc. of Glen Rock, Pa. A distal end of the substrate extends away from the sample collection end 15, forming a handle 13 for the device 10.

In operation, at least the proximal end of the device 10 is inserted into an oral cavity with the abrasive aluminum oxide surface 18 facing toward a sample collection region of the cheek. The abrasive surface 18 is placed against the sample collection region and moved vigorously and repeatedly (e.g., back and forth) along the sample surface for about 15-30 seconds. This motion abrades the skin thereby inducing a release of enriched serous fluid from the cheek into the oral cavity. The foam sponge 14 is suitably positioned to capture a sample volume of the enriched serous fluid. The sample volume of enriched serous fluid collected in the sponge 14 can be released by squeezing the saturated sponge 14 and processed further for testing purposes.

As a modification to the above embodiment, the aluminum oxide coated yellow urethane foam sponge 15 can be pretreated with about 50 microliters (about 0.0017 fl. oz.) of an aqueous solution containing 30% (wt./vol.) of citric acid (sigma) and 0.5% (wt./vol.) capsaicin.

### Example 2

In another example, referring again to FIG. 2, an alternative exemplary multilayered sample collection devices 20 includes a hook portion 28, 29 of a hook and loop fastening system, such as VELCRO Brand of Extreme Fasteners providing an aggressive closure with an all-weather adhesive specially formulated for rough surfaces, commercially available from Velcro USA Inc. of Manchester, N.H. The hook portion 28, 29 of the fastening system is cut into 7×20 mm (about 9/32× 25/32 inches) strips and adhered to a top surface of a proximal end of the substrate 12. Again, the substrate 22 is a 7×120 mm (about 9/32× 4/75 inches) strip of 0.5 mm (about 20 mills) thick strip of PET. The hook portion 28, 29 is adhered using a 7×20 mm (about 9/32× 25/32 inches) piece of double-stick acrylic adhesive tape 26a. In the exemplary embodiment, the adhesive tape 26a is as a non-sensitizing, medicinal grade wound care adhesive, such as ARCARE 8311, which is commercially available from Adhesives Research, Inc., Glen Rock, Pa. On an opposite side of the substrate 22, opposite to the hook strip 28, 29, a 7×20 mm strip of 6.4 mm (about 0.25 inch) thick urethane foam sponge 24 is attached. The sponge 24 can be attached using a 7×20 mm (about 9/32× 25/32 inches) piece of the same double-stick acrylic adhesive tape 26b as above. Once again, a distal end of the substrate 22 extends away from a sample collection end 25, forming a handle 23 for the device 20.

In operation, at least a proximal end of the device 20 is inserted into an oral cavity with the hook fastener portion 28, 29 facing toward a sample collection region of the cheek. The hook fastener portion 28, 29 is placed against the sample collection region and moved vigorously and repeatedly (e.g., back and forth) along the sample surface for about 15-30 seconds. This motion abrades the skin thereby inducing a release of enriched serous fluid from the cheek into the oral cavity. The collection device 20 is immediately removed from the mouth and reinserted with the foam sponge pad 24 placed in direct contact with the sample surface area just abraded. This results in a flow of serous fluid over a short period directly into the oral cavity followed by collection of a sample volume with the sponge 24. After about 30 seconds of serous fluid-enriched oral fluid absorption, the collection device 20 is removed and the collected sample processed.

As a modification to the above embodiment, the urethane foam sponge 24 can be pretreated with calcium oxide powder. To accomplish this, the urethane sponge 24 is placed face down onto a layer of calcium oxide against a flat surface, such as a flat pan. A thin monolayer of chemical adheres to the urethane through electrostatic interaction. The hook fastener test strip 28, 29, with adjacent calcium oxide real treated sponge pad, is then used as described above. When the calcium oxide dusted sponge comes into contact with the abraded skin, re-hydration of the chemical immediately produced heat, which in turn, increases blood flow to the cheek resulting in swelling and edema, followed by enhanced serous flow.

### Example 3

In yet another example, a multilayered sample collection device 30 formed about an elongated substrate 32 is constructed as shown in FIG. 3. The substrate is a 7×120 mm (about 9/32× 4/75 inches) strip of 0.5 mm (about 20 mills) thick PET. A 7×20 mm (about 9/32× 25/32 inches) cut strip of double-sided acrylic adhesive tape 36b. The adhesive tape 36b is a non-sensitizing, medicinal grade wound care adhesive tape, such as ARCARE 90374, also commercially available from Adhesives Research, Inc., Glen Rock, Pa. In construction, the double sided adhesive tape 36b is adhered along one side to the substrate 32 leaving a 60-pound siliconized Kraft release liner paper 38 still attached along the other side of the double-sided adhesive tape 36b. To the side adjacent to but opposite the release liner a 7×20 mm (about 9/32× 25/32 inches) piece of one 6.4 mm (about 0.25 inch) thick urethane foam 34, pretreated as described in Example 1 with a capsaicin is attached to the substrate 32 using a second double-sided adhesive strip 36b.

In operation, the release liner paper 38 is removed at the time of use, exposing an outer adhesive surface 39 of the double-sided adhesive tape 36a. The exposed adhesive surface 39 is placed between-the gums and inner cheek with the adhesive surface 39 facing a sample collection region of the cheek. An exterior surface of the same cheek, opposite to the sample collection region, is manually pressed against the adhesive surface 39 to assure adhesion of the tape 36a to the inside wall of the cheek. After about 15 seconds, the collection device 30 is rapidly pulled from the cheek removing with it several layers skin of cells. The sample collection device 30 is next reinserted into the mouth with the foam sponge 34 facing the exposed sample collection region of the cheek. Removal of the skin cells in the above manner induces serous fluid flow along a chemical gradient. Serous fluid flow is enhanced by capsaicin activity, which causes increased swelling, edema, and blood flow to the cheek. After about 30 seconds of serous fluid-enriched oral fluid absorption, the collection device 30 is removed and the collected sample processed.

In another embodiment of the sample collection device 30, the first adhesive layer 36a and release liner 38 is omitted from one side of the plastic strip substrate 32, leaving it bare. Before use, the non-coated side of the plastic strip 32 is pre-treated with 3-5 drops of a liquid adhesive solution of ethyl cyanocrylate, which is then allowed to evaporate for about 5-10 seconds. The side containing the wet adhesive is then placed in the mouth against the sample collection region of the inner cheek and held in place as described above for about 15 seconds. As with the adhesive tape 36a, the device 30 is removed from the mouth taking with it attached layers of skin cells from the sample collection region. The device 30 is reversed and placed back into the mouth to induce serous flow as described above. As before, the organic acid gradient and action of the capsaicin results in swelling, edema, and increased blood flow to the cheek resulting in heavy serous flow onto the oral fluid and collection sponge 34.

In still other embodiments of the invention, a kit includes a serous fluid collection device, such as any of the devices described above. The sample collection device is incorporated into packaging suitable for containing a medical device. Such materials include polyethylene or polypropylene, and materials made from high-density polyethylene fibers, such as those commercially available from the DuPont Company of Wilmington, Del., under the brand name TYVEK, or other types of packaging that can withstand sterilization by irradiation or gas. The kit also includes instructions for using the device. One or more of solutions of serous fluid inducers and debridement tools can also be provided separately as part of the kit. Alternatively or in addition, the one or more of solutions of serous fluid inducers and debridement tools can be pre-applied to the device. The kit can include a shipping container with postal delivery information included for shipping the used device to a predetermined remote collection or testing facility.

### Further Examples

Five abrasive materials were chosen based on grit size for use as either an abrasive sponge or non-abrasive sponge abrasive web material for oral expression of serous fluid. The abrasive sample collection devices were assembled by hot gluing a 0.5 cm (about 3/16 inch) diameter circle of die cut abrasive material onto a substrate formed from a stock no. 3774-01 wooden stick, commercially available from Wood Crafts. Table 1 provides a listing of the different abrasive materials examined for dermal abrasion efficacy.

TABLE 1
Abrasive Materials

| Condition | Description |
|---|---|
| A | Highflex 100 0115-RDR abrasive cloth |
| B | 3M Contour Sanding Sponge Medium 906NA |
| C | 3M Softback Sanding Sponge Medium |
| | 03808x20 |
| D | Mirka Abranet 9A-151-180 |
| E | Mirka Abranet 9A-151-120 |
| F | Mirka Abranet 9A-151-80 |

Each of five different volunteers were asked to supply oral samples by expectoration of a sample into a collection vessel, either following salivary stimulation alone, or after salivary stimulation coupled with dermal abrasion of the inner cheek wall. Stimulation was used to produce saliva as lubrication fluid.

All five volunteers were required to first stimulate the salivary glands by placing 20 mg (about 3 grains) of food grade citric acid into their mouths to afford lubrication. Four were instructed to cause dermal abrasion to the inside of the cheek by using a repetitive circular motion for 10 seconds, applying a substantially constant pressure against the cheek. The fifth subject was asked to apply pressure to the outside of their cheek with a finger while moving both finger and stick in the same circular motion, without causing dermal abrasion. The fifth subject served as a control providing stimulation with no abrasion even though the device was present within the oral cavity.

Samples using all six abrasive devices were collected from all volunteers over 3 days, with one collection from one cheek in the morning and another collection from the other cheek in the afternoon. The same cheek was not agitated twice within the same day or within the same position on the cheek day-to-day. All samples were stored at about −20 degrees C. (about −4 degrees F.) until tested.

All samples were tested for levels of IgA, IgG, IgM, and hemoglobin using the commercial kits listed in Table 2 below, all available from Bethyl Laboratories, Inc. of Montgomery, Tex.

TABLE 2
Sample Test Kits

| Description | Catalog No. | Lot No. |
|---|---|---|
| Human IgA ELISA Quantitation Kit | E80-102 | E-80-102-13 |
| Human IgG ELISA Quantitation Kit | E80-104 | E-80-104-16 |
| Human IgM ELISA Quantitation Kit | E80-100 | E-80-100-13 |
| Human Hemoglobin ELISA Quantitation Kit | E80-135 | E-80-135-12 |

Results of ELISA were read visually and by a color CCD to assess treatment efficacy. Results clearly indicated that both hemoglobin and IgG, IgM, and IgA relative levels rose after dermal abrasion in all subjects performing proper abrasion. In particular, Condition F listed in Table 1 (Mirka Abranet 9A-151-80 abrasive material) provided the best overall response in all individuals. The control condition of stimulation without abrasion had lower relative hemoglobin and immunoglobulin levels in all subjects and the one individual who inserted an abrasion device inter their mouth and did not abrade showed lower hemoglobin and immunoglobulin levels than properly abraded individuals and these levels were equivalent to non-abraded samples collected as controls. To quantitate IgG levels in oral fluid following proper abrasion, the procedure was repeated using Condition F. The results obtained are provided below in Table 3.

TABLE 3
Results for Repeated Procedure

| Antibody | Abrasion | O.D. | Dilution | Concentration (ng/ml) |
|---|---|---|---|---|
| IgA | Pre | 2.97 | 1:5 | 559 |
| | Post | 3.405 | 1:5 | >2500 |
| IgG | Pre | 1.908 | 1:5 | 601 |
| | Post | 3.12 | 1:5 | 2906 |
| IgM | Pre | 1.132 | 1:5 | 875 |
| | Post | 2.707 | 1:5 | 2298 |
| Hemoglobin | Pre | 0.05 | 1:5 | <31 |
| | Post | 2.206 | 1:5 | >5000 |

The results indicate a three to four fold increase in immunoglobulin concentrations measured in ng/ml following abrasion with Mirka Abranet 9A-151-080 (Condition F) and an increase in hemoglobin levels of over 150 fold. Comparable quantitative results were observed in all subjects tested. After visual inspection, none of the samples from any of the volunteers were bloody in appearance.

The device described provides a novel method of detecting an analyte or substrate in a mammal, comprising: obtaining a sample of serous fluid from the mammal using the device (or the kit), and detecting the analyte or substrate in the serous fluid of the mammal. The analyte or substrate in the serous fluid of the mammal may indicate the presence of an infection in the mammal, such as a viral infection including HIV, HPV, herpes virus, hepatitis A, B or C, influenza, Ebola, West Nile Virus, Dengue Fever, or another communicable viral infection. The infection so detected may also be a bacterial infection.

Various embodiments of improved devices and methods for orally sampling serous fluid have been described herein. These embodiments are given by way of example and are not intended to limit the scope of the present invention. Other embodiments of this invention will be apparent to those skilled in the art upon consideration of this specification or from practice of the invention disclosed herein. Various omissions, modifications, and changes to the principles and embodiments described herein may be made by one skilled in the art without departing from the true scope and spirit of the invention. It should be appreciated, moreover, that the various features of the embodiments that have been described may be combined in various ways to produce numerous additional embodiments. Moreover, while various materials, dimensions, shapes, materials, and compounds have been described for use with disclosed embodiments, others besides those disclosed may be utilized without exceeding the scope of the present disclosure as recited in the following claims.

What is claimed is:

1. An oral serous fluid collection device comprising:
a substrate configured for placement within an oral cavity;
a sample collector attached to the substrate and configured to collect a sufficient sample volume for performing a target assay; and
at least one sample inducer disposed on at least one of the substrate and the sample collector, the sample inducer configured to induce a flow of serous fluid from an oral surface.

2. The device of claim 1, wherein the sample inducer includes effective amounts of one or more compounds that induce serous fluid flow at the site of application.

3. The device of claim 2, wherein the one or more compounds include dilute organic acids such as acetic acid, malic acid, or citric acid or similar acids, urea, and where the one or more compounds establish a chemical or osmotic gradient at the site of application.

4. The device of claim 2, wherein the one or more compounds include desiccants, establishing a moisture gradient at the site of application.

5. The device of claim 4, wherein the desiccants are selected from the group consisting of: ceramic micro-particles; ATS; zeospheres; silica gel; desiccating paper; hydrophilic silica nano-particles; absorbent polymers, such as cross-linked agaorose or acrylamide or starch co-polymers; and combinations thereof.

6. The device of claim 2, wherein the one or more compounds include chemical agents configured to exhibit an exothermic reaction upon hydration by contact with an endothelial or dermal surface at the site of application.

7. The device of claim 2, wherein the chemical agents are selected from the group consisting of: calcium oxide; calcium sulfate; calcium carbonate; calcium chloride; sodium acetate; and combinations thereof, wherein the compounds are configured to induce one or more of inflammation, swelling and chemical burn of the endothelial or dermal surface.

8. The device of claim 2, wherein the one or more compounds include capsaicin or capsaicinoids including one or more of dihydrocapsaicin, nordihydrocapsaicin, homodihydrocapsaicin, and homocapsaicin, the one or more compounds inducing inflammation, swelling and serious fluid expression at the site of application.

9. The device of claim 2, wherein the sample inducer further comprises an anesthetic agent.

10. The device of claim 9, wherein the anesthetic agent is selected from the group consisting of: menthol; camphor; lidocaine; prilocaine; benzocaine; butacaine; cyclomethicaine; dibucaine; tetracaine; an equivalent anesthetic agent; and combinations thereof.

11. The device of claim 2, wherein the sample inducer comprises a vasodilator compound, including one or more of histamine, bradykinin, 5-HT, PAF, and prostaglandins.

12. The device of claim 2, wherein the sample inducer comprises a parasympathetic nervous system stimulator compound, including pilocarpine.

13. The device of claim 2, wherein the sample inducer further comprises an enzyme.

14. The device of claim 2, wherein the enzyme is selected from the group consisting of: trypsin; chymotrypsin; elastase; proteinase K; papain; collagenase; serine proteases; enzymes that hydrolyze ester or peptide bonds; and combinations thereof.

15. The device of claim 2, wherein the sample inducer includes at least two agents including collagenase, proteinase K, papain and urea.

16. The device of claim 1, wherein the device further includes a cell removing region.

17. The device of claim 16, wherein the cell removing region and the sample inducer are contiguous, overlapping, interspersed or otherwise co-localized.

18. The device of claim 16, wherein the cell removing region provides a mechanically abrasive surface.

19. The device of claim 18, wherein the mechanically abrasive surface comprises fixed abrasive particles having a high grit size, the abrasive particles including one or more of aluminum oxide, calcined alumina oxide, silicon carbide, zirconia alumina, crushed glass, crocus and similar abrasives.

20. The device of claim 16, wherein the cell removing region comprises physical structures having a hook morphology placed on the device to come into contact with the oral surface.

21. The device of claim 16, wherein the cell removing region comprises physical structures having a sharp or tapered morphology.

22. The device of claim 21, wherein the sharp or tapered physical structures are selected from the group consisting of: files, grates, brushes, projections, microprojections; and combinations thereof.

23. The device of claim 16, wherein the cell removing region comprises an adhesive layer configured to adhere to the oral surface and remove one or more cell layers when removed therefrom.

24. The device of claim 1, wherein the sample collector comprises an absorptive inert material.

25. The device of claim 24, wherein the absorptive inert material is selected from the group consisting of: high density hydrophilic polypropylene; polyurethane foam; glass fiber membrane; aluminum oxide membrane; polycarbonate; polyester; PVDF; polyolefin; PTFE; aluminum; glass; other inert matrices; absorptive non-inert materials including cellulosic membranes; and combinations thereof.

26. The device of claim 1, wherein the sample collector comprises adsorptive inert material.

27. The device of claim 26, wherein the adsorptive inert material is selected from the group consisting of: zeolite films; silica gel containing papers; nylon; an adsorptive non-inert media including nitrocellulose; an adsorptive non-inert media including nitrocellulose cellulose nitrate; and combinations thereof.

28. A method for collecting an enriched serous fluid sample from a mammalian oral cavity comprising:
placing a sample inducer against an adjacent surface of the oral cavity;
inducing with the sample inducer an flow of enriched serous fluid from the oral surface; and
collecting a sample volume of the induced, enriched serous fluid, wherein the sample volume is sufficient for performing a target assay.

29. The method of claim 28, further comprising removing a tissue layer prior to collecting the sample volume.

30. The method of claim 28, wherein inducing comprises applying effective amounts of one or more compounds that induce serous fluid flow at the site of application.

31. The method of claim 28, further comprising applying a topical anesthetic agent to the adjacent surface of the oral cavity.

32. The method of claim 28, wherein inducing comprises rubbing a mechanically abrasive surface against the adjacent surface of the oral cavity.

33. The method of claim 28, wherein collecting the sample volume comprises absorbing a sufficient volume of induced, enriched serous fluid.

34. The method of claim 28, further comprising the step of adding to the collected sample volume a preservative compound.

35. The method of claim 28,-further comprising the step of performing an assay using the collected sample volume.

36. A serous fluid collection kit comprising:
a serous fluid collection device according to claim 1;
sealable packaging for storing the serous fluid collection device; and
instructions for using the device.

37. A method of detecting an analyte or substrate in a mammal, comprising: obtaining a sample of serous fluid from the mammal using the device of claim 1, and detecting the analyte or substrate in the serous fluid of the mammal.

38. The method of claim 37, wherein the analyte or substrate in the serous fluid of the mammal indicates the presence of an infection in the mammal.

39. The method of claim 38, wherein the infection is a viral infection including HIV, HPV, hepatitis A, B or C, influenza, or a communicable viral infection.

40. The method of claim 36, wherein the infection is selected from the group of infections consisting of: bacterial; chylamdial; rickettsial; fungal; mycoplasmal; protozoal; helminthal; and combinations thereof.
